(19) United States
(12) Patent Application Publication
(10) Pub. No.: US 2010/0324636 A1
(43) Pub. Date: Dec. 23, 2010

(54) MEDICAL IMPLANTABLE LEAD WITH PIVOTING SEGMENTS (As Amended)

(76) Inventors: Rolf Hill, Jarfalla (SE); Olof Stegfeldt, Alta (SE)

Correspondence Address: SCHIFF HARDIN, LLP;PATENT DEPARTMENT, 233 S. Wacker Drive-Suite 6600, CHICAGO, IL, 60606-6473, US

(21) Appl. No.: 12/599,562

(22) PCT Filed: May 15, 2007
(86) PCT No.: PCT/SE07/00471

§ 371 Date: Nov. 10, 2009

(51) Int. Cl. A61N 1/00

(52) U.S. Cl. 607/116

(57) ABSTRACT

A medical implantable lead, which is adapted to be attached with a distal end to tissue inside a human or animal body, has a distal end that is variable in size between an introducing state, when the distal end has a minimum surface area, and a mounting state when the surface area of the distal end is enlarged in relation to its minimum surface area. For this purpose, the distal end of the medical implantable lead has several pivoting segments, each being pivotally hinged about a pivot axis directed substantially tangentially in relation to the lead, with each pivoting segment being pivotable about the pivot axis between an introducing state in which each pivoting segment is rotated to a position in parallel or in a small angle to the longitudinal axis of the lead, and a mounting state in which each pivoting segment is rotated to a position essentially perpendicular to the longitudinal axis of the lead.

### BACKGROUND OF THE INVENTION

1. Field of the Invention

The invention relates to a medical implantable lead, which is adapted to be attached with a distal end to tissue inside a human or animal body, of the type wherein the distal end is variable between an introducing state, when the distal end has a minimum surface area, and a mounting state when the surface area of the distal end is enlarged in relation to its minimum surface area.

2. Description of the Prior Art

Medical implantable leads of various kinds and for various applications, e.g. for monitoring and controlling the heart in a human or animal body by means of a pacemaker, tend to become smaller and smaller in cross section. A medical implantable lead for pacemaker applications, for example, can have a diameter of less than 2 mm. This is advantageous in one aspect, since then the lead will be more flexible and take up less space. However, there are also risks with leads having a too small cross sectional dimension. In many cases the leads are namely adapted to be mounted to an organ inside the body, e.g. a heart wall, with its end surface abutting the organ and held by means of for example a helix, which is screwed into the organ. If the cross sectional dimension of the lead is too small in relation to its stiffness, it is a risk that the lead tip will perforate the organ during mounting of the lead and cause bleeding. This risk exists on the one hand when the distal end is pressed against the organ but before the lead is actually attached to it. In case the lead is provided with a rotatable helix for attaching to the organ, this risk also exists when the helix is screwed into the organ such that the distal end is drawn into the tissue by means of the rotating action of the helix. When the lead is attached to e.g. a heart, which performs large movements during function, there is also a risk that the heart wall will be perforated or injured during the course of a longer period of time when the lead is attached, due to abrasion or the like, if the lead is made with a too small cross sectional dimension in relation to its stiffness.

From United States Patent Application Publication No. 2007/0050003 A1, is known a medical implantable lead, which in one embodiment (FIGS. 7A-C) is provided with an elastic sleeve in a distal end which protrudes a distance beyond a distal end of the rest of the lead and which, when abutting the distal end against tissue or attaching the lead to the tissue by means of screwing a helix into the tissue, will buckle and expand to a diameter that is larger than the diameter of the distal end of the lead itself. However, there are several disadvantages with a medical implantable lead of this kind. To accomplish buckling of the sleeve, it is required a rather large force having to effect that, when the lead is mounted with its distal end in abutment against tissue, the sleeve will affect the lead with a force striving to push the lead from and disengage the helix from the tissue. This is not satisfactory since it may lead to that the helix might slip out of engagement with the tissue during long term usage due to movements in the tissue, e.g. a heart. To alleviate this effect, one possible solution would be to make the sleeve extremely elastic and flexible, but this would also have to effect that the contribution of the sleeve for preventing perforation into the tissue, would be reduced correspondingly. It would also have to effect that the risk for unintentional buckling of the sleeve, and hence an increased lead tip area, when introducing the lead through e.g. a narrow vein will increase, which might make it impossible to insert the lead.

### SUMMARY OF THE INVENTION

It is an object of the invention to provide an improved medical implantable lead with which the risk of perforation into tissue is eliminated or reduced and that still can ensure secure attachment to tissue.

The basis of the invention is the insight that the above object may be achieved by providing the medical implantable lead with a mechanism that varies (changes) the surface area of the distal end of the lead, such that the surface area has a minimum when introducing the lead into the body. Once the lead is localized inside the body, the surface area of the distal end can be enlarged when abutting the distal end against an organ or other tissue.

According to the invention, the enlargement of the surface area is accomplished by mechanically pivoting of material portions at the distal end. More precisely, the distal end of the lead is provided with several pivoting segments distributed around its circumference, each of the pivoting segments being pivotably hinged about a pivot axis directed substantially tangentially in relation to the lead. Preferably, the pivot axis is located on the inside close to the center of each pivoting segment. Each pivoting segment is pivotable about the pivot axis between a first, introducing position, when each pivoting segment is rotated backward/outward to a position being in close contact with the outer surface of the lead and in parallel or in a small angle to the longitudinal axis of the lead, and a second, mounting position when each pivoting segment is rotated to a position essentially perpendicular to the longitudinal axis of the lead. With a medical implantable lead arranged in this way, no force is required to assume the mounting state with an enlarged surface area, also having to effect that no force is acting on the lead from the pivoting segments striving to push the lead away from the tissue. There is also no risk that the mounting state, with an enlarged surface area, is unintentionally assumed during introduction of the lead through a vein or the like.

In the embodiment described and illustrated below, the articulated hinge between the distal end of the lead and the pivoting segments, is accomplished by a sleeve of an elastic material, e.g. silicon plastics, which covers the outside of the lead, and the pivoting segments are formed of the same material as and in one unitary piece with the sleeve, and such that they are connected together by a thin tongue of the elastic material having to effect that the pivotable action of the pivoting segments are achieved by elastic deformation of the tongue. However, it also would be possible to form the pivoting segments as separate objects, in relation to the rest of the lead, and to connect them via a mechanical hinge to the lead, e.g. by snap fitting.

Preferably, the lead according to the invention also has an extracting state in which each pivoting segment is rotated forward/inward such that an outer edge of each pivoting segment is positioned further in a distal direction than an inner edge. In this way the surface area of the distal end will be reduced in relation to the mounting state to facilitate extraction of the medical implantable lead from the human or animal body.

It is to be understood that the medical implantable lead may be modified in many different ways in relation to the hereinafter described and illustrated embodiment. E.g. the ring segments may have many different shapes than the herein showed ring segment shape, though it is normally advantageous if the segments have a generally flat shape.

### BRIEF DESCRIPTION OF THE DRAWINGS

FIG. 1 is a perspective view of the distal end of a medical implantable lead according to the invention, in a mounting state when the pivotable segments have been rotated to a position substantially perpendicular to the longitudinal axis of the lead.

FIG. 2 is a perspective view of the outermost distal end of the lead of FIG. 1 in an introducing state, when the pivotable segments have been rotated backwardly/outwardly.

FIG. 3 is a perspective view of the outermost distal end of the lead of FIGS. 1 and 2 in an extracting state, when the pivotable segments have been rotated forwardly/inwardly.

FIG. 4 is an enlarged longitudinal section through the distal end of the lead of FIGS. 1 through 3.

FIG. 5 is a longitudinal section through the distal end of the lead when being introduced through a vein.

FIG. 6 is a longitudinal section of the lead of FIG. 5 in a mounted state, with the distal end abutting a heart wall.

FIG. 7 is a longitudinal section of the lead according to FIGS. 5 and 6, with the lead in any extracting state being extracted from the body through a vein.

### DESCRIPTION OF THE PREFERRED EMBODIMENTS

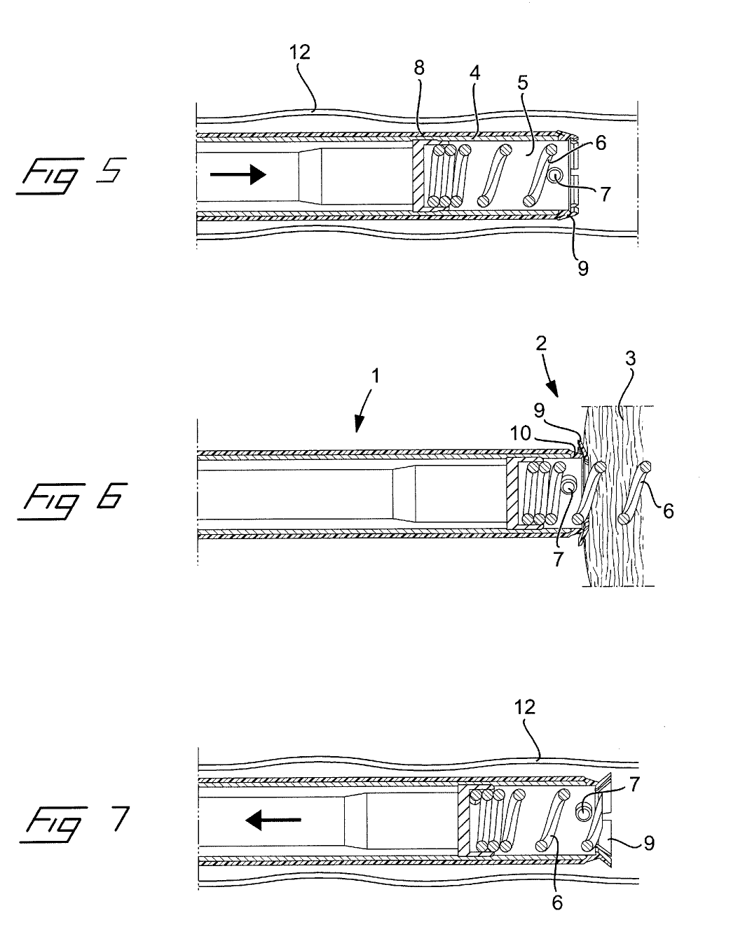

The medical implantable lead according to an embodiment of the invention and being illustrated in the drawings, is adapted to be used in connection with a pacemaker or an implantable cardioverter defibrillator. Accordingly, the lead comprises a considerably long electrical lead 1, of which only a distal end is shown in the drawings. A pacemaker is adapted to be connected to a proximal end of the lead, whereas a distal end 2 is adapted to be attached to a heart wall 3. As is best seen in FIGS. 4-7, the lead is, in a distal end, provided with a rigid tubular header 4 of a metal or plastics. In a distal end of a bore 5 in the header, a helix 6 is rotatably arranged. The helix can be rotated from the proximal end of the lead, by suitable means known in the art but not shown in the drawings, such as a stylet or a coil. Besides being rotatably, the helix 6 is also displaceable in an axial direction in the header 4. This is achieved by the helix winding being in engagement with a post 7 on the inside of the header, such that when rotating the helix, it will also be displaced in the axial direction of the lead. This is illustrated in FIG. 5, where the helix 6 is screwed in and completely accommodated within the header bore 5, and in FIG. 6 where the helix is screwed out such that a distal portion of the helix protrudes a distance out from the header and extended into the tissue of a heart wall 3. On the outside of the header the lead is provided with a cover or sleeve 8 of a resilient material, e.g. of silicone.

As is illustrated, the distal end of the medical implantable lead according to the invention, is provided with several pivoting segments 9. In the enlargement of FIG. 4, it can be seen that the pivoting segments are formed of the same material and in a unitary peace with the elastic sleeve 8 on the outside of the header. Each pivoting segment is formed as a ring segment, having a generally flat shape, and is pivotable by elastic deformation of a tongue 10, which connects the pivoting segment 9 with the elastic sleeve 8. Accordingly, each segment is individually pivotable in relation to the header 4. The illustrated lead is provided with six separate segments and it is believed that at least four segments are required for a proper function.

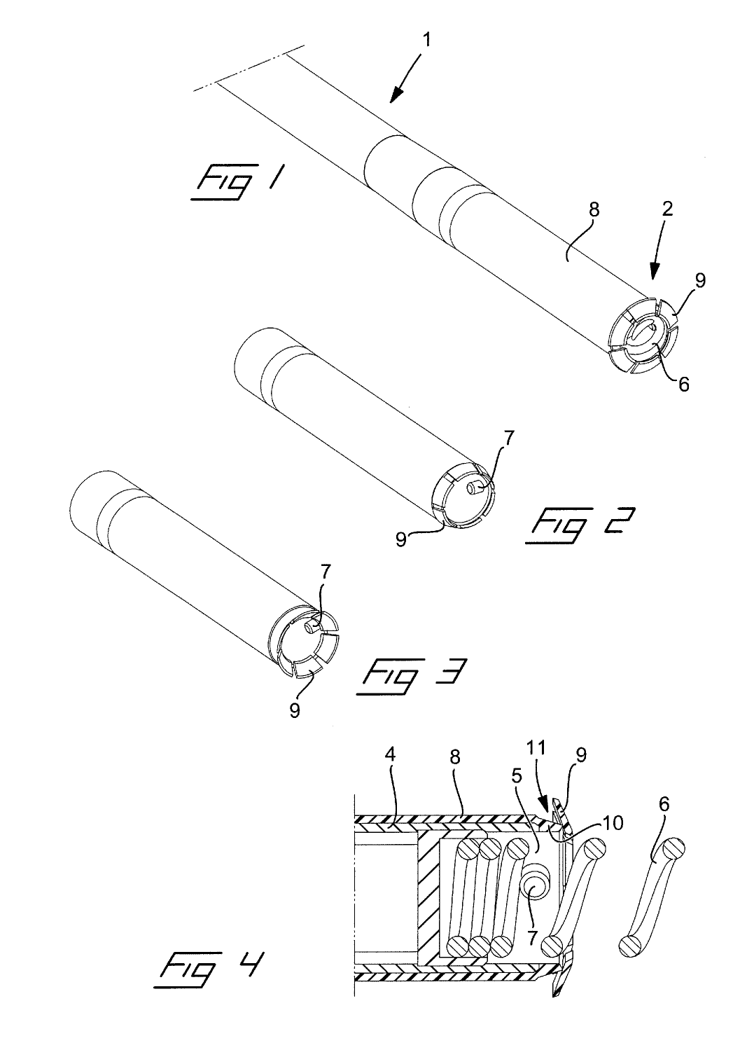

From FIG. 4 is also evident that the pivoting segments 9 are connected to the elastic sleeve 8 in an area near the middle of each segment but displaced towards the inner periphery of the tubular header 4, and the sleeve is provided with a recessed or beveled portion 11 on the outer side of the distal end. The latter is due to the fact that preferably no part of the segments should protrude beyond the outer boundaries of the lead in the introducing state according to FIG. 5. The recessed portion 11 has a depth that is at least as large as the thickness of the segments such that the segments can be accommodated in the recessed area in the introducing state. In this way the pivoting segments 9 may be pivoted backward/outward and accommodated in the recessed portion 11 such that essentially no part of the pivoting segments protrude outside the outermost periphery of the lead when the lead is in the introducing state according to FIGS. 2 and 5.

Preferably, the initial position of the pivoting segments 9, when no forces are effecting them, is as is illustrated in FIG. 1, i.e. the pivoting segments are positioned with their principal plane substantially perpendicular to the longitudinal axis of the lead. Accordingly, the segments will adopt this position as soon as the distal end comes into a cavity in a body. This position will also be adopted as soon as the distal end of the lead comes into contact with a surface, e.g. a wall 3 of a heart, when the helix is rotated and screwed into tissue for attaching the lead to the tissue. In this way the surface area of the lead is increased such that the risk for penetration of the lead into tissue is reduced. When the lead is introduced into a human or animal body, through a vein 12 or the like, the pivoting segments assume an introducing state with the segments pivoted backward/outward with only a small angle in relation to the longitudinal axis of the lead, as is illustrated in FIGS. 2 and 5.

When extracting the lead from the body, the pivoting segments 9 will adopt the position illustrated in FIG. 7. Namely, when the lead encounter resistance from e.g. the inside of a vein 12, the outer edges of the segments will slide against the walls of the vein 12 and be pivoted forward/inward such that the outer edges of the segments will be positioned further in the distal direction in relation to the inner edges. This situation may occur several years after implanting of the lead into the body and by that time, the distal end can be more or less overgrown by tissue. In this way the lead can be extracted with a reduced risk for damage to or entangle in tissue.

When implanting the lead into a human or animal body, e.g. by introducing it through a vein 12, the lead is in the introducing state as is shown in FIGS. 2 and 5. In this state the helix is screwed into and accommodated in the header bore 5, which prevents the helix from penetration into tissue during introduction. When the lead has been introduced into the body and the distal end of the lead, with the pivoting segments 9 in a mounting state substantially perpendicular to the longitudinal axis, is bearing against tissue at a location to be mounted in, the helix 6 is caused to rotate by means of a not shown rotary transmitting means, such as a stylet or a coil, by means of which a rotary movement can be transmitted from a proximal end to the distal end of the lead. When rotating the helix, its tip will penetrate into the tissue 3 and attach the lead securely to the tissue, as shown in FIG. 6.

Although modifications and changes may be suggested by those skilled in the art, it is the intention of the inventors to embody within the patent warranted heron all changes and modifications as reasonably and properly come within the scope of their contribution to the art.

We claim as our invention:

1-8. (canceled)

9. A medical implantable lead, comprising:
a lead body configured for in vivo implantation in a patient, said lead body having a distal end adapted for in vivo attachment to tissue in the patient;
said distal end having a shape that is variable, by application of a force thereto, between an introducing state shape, in which the distal end has a minimum surface area, and a mounting state shape, in which a surface area of the distal end is enlarged relative to said minimum surface area;
said distal end comprising a plurality of pivotable segments, each pivotable segment being hinged for pivoting around a pivot axis oriented substantially tangentially relative to said lead body;
each pivotable segment being pivotable by said force around said pivot axis between said introducing state, in which each pivotable segment is rotated to a position substantially parallel to a longitudinal axis of said lead body, and said mounting state in which each pivotable segment is pivoted to a position substantially perpendicular to said longitudinal axis.

10. A medical implantable lead as claimed in claim 9 wherein each of said pivotable segments is comprised of the same material in a unitary piece with an elastic sleeve on an exterior of said lead body, and wherein said pivotable segments are hinged by an elastic tongue extending between said elastic sleeve and said segments.

11. A medical implantable lead as claimed in claim 9 wherein said pivoting segments are formed as ring segments.

12. A medical implantable lead as claimed in claim 9 wherein said distal end of said lead body comprises a recessed or beveled portion on an exterior side thereof that accommodates said pivotable segments in said introducing state.

13. A medical implantable lead as claimed in claim 9 wherein said pivotable segments are hinged to said lead body at a position localized toward an inner periphery of a tubular header of said lead body.

14. A medical implantable lead as claimed in claim 9 comprising four of said pivotable segments.

15. A medical implantable lead as claimed in claim 9 wherein said distal end has an extracting state in which said surface area of said distal end is reduced relative to said mounting state to facilitate extraction of said lead body from said patient.

16. A medical implantable lead as claimed in claim 15 wherein, in said extracting state, said pivotable segments are rotated forwardly and inwardly relative to said lead body.
